(19) United States
(12) Patent Application Publication
(10) Pub. No.: US 2020/0258617 A1
(43) Pub. Date: Aug. 13, 2020

(54) TRANSFER DESTINATION DETERMINATION SYSTEM, TRANSFER DESTINATION DETERMINATION METHOD, AND TRANSFER DESTINATION DETERMINATION PROGRAM

(71) Applicant: NEC CORPORATION, Tokyo (JP)

(72) Inventors: Masahiro HAYASHITANI, Minato-ku, Tokyo (JP); Masahiro KUBO, Minato-ku, Tokyo (JP)

(73) Assignee: NEC CORPORATION, Tokyo (JP)

(21) Appl. No.: 16/756,637

(22) PCT Filed: Sep. 20, 2018
(86) PCT No.: PCT/JP2018/034847

§ 371 Date: Apr. 16, 2020

(30) Foreign Application Priority Data
Oct. 17, 2017 (JP) 2017-200719

(51) Int. Cl. G16H 40/20; G16H 10/60; G16H 50/20; G16H 50/50; G06Q 10/02

(52) U.S. Cl. CPC G16H 40/20; G16H 10/60; G06Q 10/02; G16H 50/50; G16H 50/20

(57) ABSTRACT

A first prediction unit 81 predicts a transfer destination of a target patient, based on inputted information on the target patient and a first prediction model for prediction of a transfer destination of a patient. A second prediction unit 82 predicts a treatment completion period of the target patient, based on information on the target patient and a second prediction model for prediction of a treatment completion period of a patient. An acquisition unit 83 acquires facility information including an operation status of each facility. A determination unit 84 determines a facility that satisfies a requirement of a transfer destination from among the facilities, based on the facility information that has been acquired and the transfer destination and the treatment completion period that have been predicted. An output unit 85 outputs a result determined by the determination unit 84.

### TECHNICAL FIELD

The present invention relates to a transfer destination determination system, a transfer destination determination method, and a transfer destination determination program for determining a transfer destination of a patient.

### BACKGROUND ART

There are situations where a movement destination of a patient is determined from the viewpoint of changes in a patient's condition, hospital equipment, and the like. For example, PTL 1 describes a cooperation method in a case where a patient who is undergoing treatment is moved from a present institution to another institution to have re-treatment. In the method described in PTL 1, when treatment at the present institution is interrupted, suitability of other institutions is evaluated and another institution capable of providing treatment is selected so that treatment of the same or with allowable difference can be continued.

Note that PTL 2 describes a facility reservation management system capable of reserving a facility and equipment via a network. The system described in PTL 2 controls reservation contents such as a date and time, a purpose, and a fee related to the facility reservation, and a user reserves or cancels the reservation on the basis of the control.

### CITATION LIST

### Patent Literature

PTL 1: Japanese Patent Application Laid-Open No. 2010-148534

PTL 2: Japanese Patent Application Laid-Open No. 2005-182425

### SUMMARY OF INVENTION

### Technical Problem

After a patient's condition in an acute period becomes stable and enters a recovery period, it is desirable to be able to determine an appropriate destination for the patient, also from the viewpoint of reducing a cost burden of the patient and improving efficiency of hospital management. However, even if an attempt is made to reserve a movement destination facility immediately before discharge from a hospital, there is a possibility that the reservation cannot be made. Therefore, it is preferable to make a reservation in advance, but it is also difficult to hold a reservation of a facility for a long time.

However, a patient's condition and a treatment state are highly uncertain, and a state of the movement destination is also changing every moment. For example, the method described in PTL 1 does not consider a current state of other institutions. Therefore, timing of moving to another institution that has been determined is unclear and it is not always possible to move, which makes it difficult to shorten a hospital stay duration of the patient.

Therefore, an object of the present invention is to provide a transfer destination determination system, a transfer destination determination method, and a transfer destination determination program capable of determining a transfer destination so as to shorten a hospital stay duration of a patient.

### Solution to Problem

A transfer destination determination system according to the present invention includes: a first prediction unit that predicts a transfer destination of a target patient, based on inputted information on the target patient and a first prediction model for prediction of a transfer destination of a patient; a second prediction unit that predicts a treatment completion period of the target patient, based on information on the target patient and a second prediction model for prediction of a treatment completion period of a patient; an acquisition unit that acquires facility information including an operation status of each facility; a determination unit that determines a facility that satisfies a requirement of a transfer destination from among the facilities, based on the facility information that has been acquired and the transfer destination and the treatment completion period that have been predicted; and an output unit that outputs a result determined by the determination unit.

A transfer destination determination method according to the present invention: predicts a transfer destination of a target patient, based on inputted information on the target patient and a first prediction model for prediction of a transfer destination of a patient; predicts a treatment completion period of the target patient, based on information on the target patient and a second prediction model for prediction of a treatment completion period of a patient; acquires facility information including an operation status of each facility; determines a facility that satisfies a requirement of a transfer destination from among the facilities, based on the facility information that has been acquired and the transfer destination and the treatment completion period that have been predicted; and outputs a determined result.

The transfer destination determination program according to the present invention causes a computer to execute: a first prediction process of predicting a transfer destination of a target patient, based on inputted information on the target patient and a first prediction model for prediction of a transfer destination of a patient; a second prediction process of predicting a treatment completion period of the target patient, based on information on the target patient and a second prediction model for prediction of a treatment completion period of a patient; an acquisition process of acquiring facility information including an operation status of each facility; a determination process of determining a facility that satisfies a requirement of a transfer destination from among the facilities, based on the facility information that has been acquired and the transfer destination and the treatment completion period that have been predicted; and an output process of outputting a result determined in the determination process.

### Advantageous Effects of Invention

According to the present invention, a transfer destination can be determined so as to shorten a hospital stay duration of a patient.

### BRIEF DESCRIPTION OF DRAWINGS

FIG. 1 It depicts a block diagram showing an exemplary embodiment of a transfer destination determination system according to the present invention.

FIG. 2 It depicts an explanatory view showing an example of patient information.

FIG. 3 It depicts an explanatory view showing an example of transfer destination information.

FIG. 4 It depicts an explanatory view showing an example of a method for predicting the number of days until completion of treatment.

FIG. 5 It depicts an explanatory view showing an example of another method for predicting the number of days until completion of treatment.

FIG. 6 It depicts an explanatory view showing an example of a facility reservation in consideration of a prediction difference.

FIG. 7 It depicts a flowchart showing an operation example of the transfer destination determination system.

FIG. 8 It depicts a block diagram showing a modified example of the transfer destination determination system according to the present invention.

FIG. 9 It depicts a block diagram showing an outline of the transfer destination determination system according to the present invention.

### DESCRIPTION OF EMBODIMENTS

If a hospital stay duration of a patient can be shortened, a cost burden on the patient side can be reduced, and a hospital side has also an advantage that it is easier to accept patients in an acute period. In addition, a facility on the accepting side can also grasp information on the patient to be accepted in advance, and as a result, it is also possible to perform operations such as preparation for the acceptance and adjustment of personnel in advance.

Hereinafter, an exemplary embodiment of the present invention will be described with reference to the drawings.

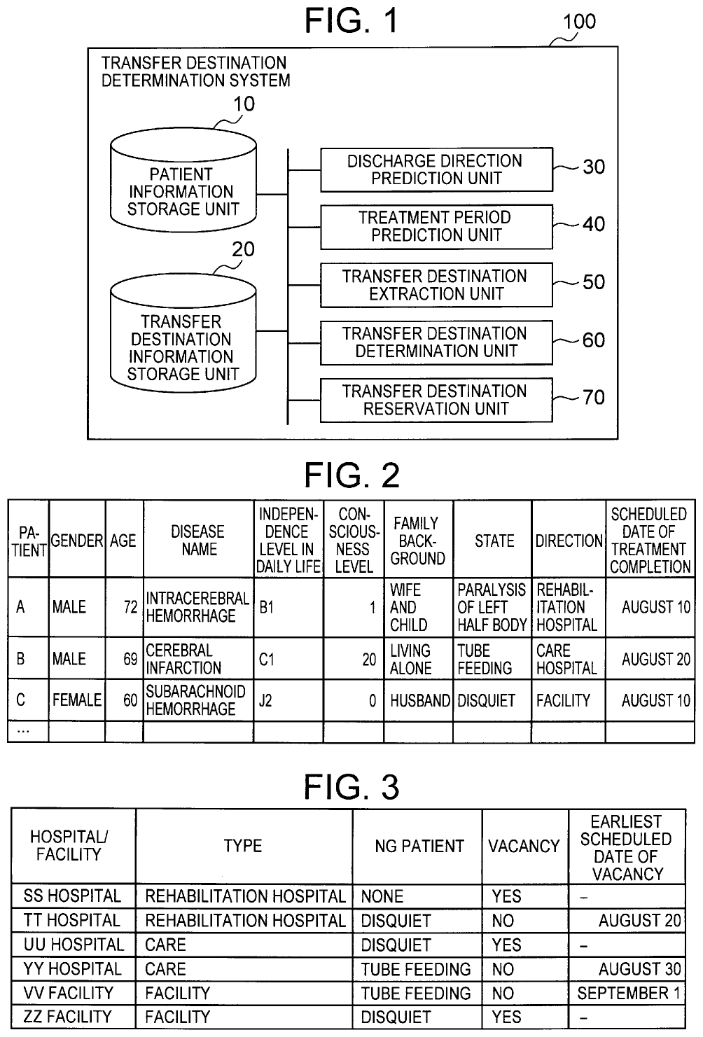

FIG. 1 is a block diagram showing an exemplary embodiment of a transfer destination determination system according to the present invention. A transfer destination determination system 100 of the present exemplary embodiment includes a patient information storage unit 10, a transfer destination information storage unit 20, a discharge direction prediction unit 30, a treatment period prediction unit 40, a transfer destination extraction unit 50, a transfer destination determination unit 60, and a transfer destination reservation unit 70.

The patient information storage unit 10 stores target patient information. The patient information storage unit 10 may store, for example, electronic medical chart data as the patient information. FIG. 2 is an explanatory view showing an example of patient information. The patient information exemplified in FIG. 2 includes an independence level in daily life, a consciousness level according to the Japan Coma Scale (JCS), a state, and the like, in addition to gender, age, a disease name, and a family background. Further, the patient information storage unit 10 may store a treatment completion period predicted by the treatment period prediction unit 40, which will be described later, or a discharge direction predicted by the discharge direction prediction unit 30.

In addition, the patient information storage unit 10 may store related information such as a place of patient's residence, as the patient information. Further, the patient information storage unit 10 may store not only information on the patient itself but also a place of residence of a caregiver of the patient, as the patient information.

The transfer destination information storage unit 20 stores information on candidates for a transfer destination. Note that, in this specification, the term “transfer” is used to have a meaning including a hospital transfer (or doctor change). Further, the transfer destination is to indicate a destination place (facility) to which a patient moves from a place where the patient is hospitalized (for example, a destination hospital of emergency transportation). Examples of the transfer destination include, for example, a home, a medical care hospital or ward, a hospital or ward providing rehabilitation, a nursing facility, and the like. However, the transfer destination is not limited to the example described above.

Further, in the present exemplary embodiment, a transfer destination that requires a reservation is referred to as a facility. The facility is a medical facility such as a hospital in a narrow sense, but the form of the facility is not limited to a medical facility, and may be, for example, an accommodation facility allowing recuperation.

The transfer destination information storage unit 20 stores, as information on a facility to be the transfer destination, a type of the facility, a type of a patient whose acceptance is difficult, and an operation status, for each facility. Here, the type of the facility indicates a type of a facility for supporting an action required after the patient is discharged from the hospital. Examples of the type of the facility include the above-mentioned medical care hospital (hereinafter, also referred to as a care hospital), a hospital providing rehabilitation (hereinafter, also referred to as a rehabilitation hospital), a nursing facility, and the like. In addition, since a transfer destination facility indicates a direction after discharge of the patient, the type of facility can be referred to as a discharge direction.

Further, the operation status indicates a status in which a patient can be accepted. The operation status includes, for example, whether or not there is a vacancy in the facility, an earliest scheduled date of a vacancy at the present moment, and the like. In addition, the operation status may include the number of vacant beds, the number of people that can be accepted, and the like.

FIG. 3 is an explanatory view showing an example of a transfer destination information. The example shown in FIG. 3 shows that the transfer destination information storage unit 20 stores, for each facility, a type, a patient whose acceptance is difficult (NG patient), a vacancy status of the facility, and an earliest scheduled date of a vacancy.

The transfer destination information storage unit 20 may additionally store information such as a location condition, a medical expense, reception hours, and medical hours as information on the transfer destination facility.

The discharge direction prediction unit 30 inputs information on a target patient, and predicts a transfer destination of the target patient on the basis of the inputted information on the patient and on the basis of a model (hereinafter, referred to as a first prediction model) for prediction of a transfer destination of the patient. Note that, in the present exemplary embodiment, it is assumed that the first prediction model has been learned in advance and stored in a storage unit (not shown).

Any form of the first prediction model may be adopted. The first prediction model may be, for example, a prediction model in which a category of a discharge direction of the patient (for example, discharged to home, transferred to a rehabilitation hospital, transferred to a care hospital, entering a nursing facility, and the like) is used as a target variable, and an item of the patient information exemplified in FIG. 2 is used as an explanatory variable.

In addition, the discharge direction prediction unit 30 may predict the transfer destination of the patient by using a plurality of prediction models for determining suitability of the discharge direction of the patient described above. For example, when this prediction model outputs a degree of suitability as a prediction result, the discharge direction prediction unit 30 may select a transfer destination considered to be the most suitable, from the prediction result.

The treatment period prediction unit 40 predicts a treatment completion timing for a patient. Specifically, the treatment period prediction unit 40 inputs information on the target patient, and predicts a treatment completion period for the target patient on the basis of the inputted information on the patient and a model (hereinafter, referred to as a second prediction model) for prediction of a treatment completion period of a patient. In the present exemplary embodiment, it is assumed that the second prediction model has been learned in advance and stored in a storage unit (not shown).

Here, the treatment completion period is a period of a predicted number of days until completion of treatment (or a treatment completion date) with a certain allowance. In general, it is difficult to predict a date at a pinpoint. Therefore, in the present exemplary embodiment, the treatment period prediction unit 40 predicts the number of days until completion of treatment in consideration of a certain allowance.

Any form of the second prediction model may also be adopted. A target to be predicted by the second prediction model is the number of days until completion of treatment (or a treatment completion date). Therefore, for example, as the second prediction model, a prediction model is considered in which the number of days until completion of treatment is used as a target variable and an item of patient information exemplified in FIG. 2 is used as an explanatory variable.

However, it is difficult to improve accuracy of predicting the number of days until completion of treatment as described above. Therefore, the treatment period prediction unit 40 may predict the number of days until completion of treatment by performing multi-class classification by using a plurality of prediction models.

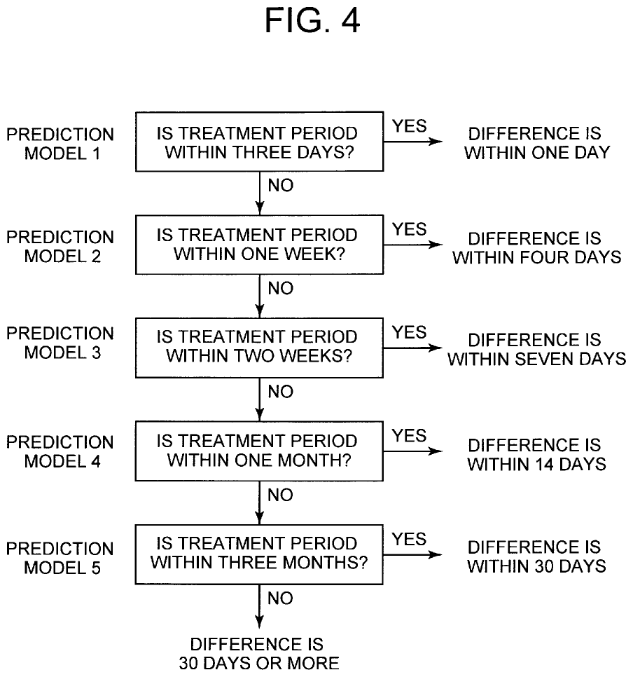

FIG. 4 is an explanatory view showing an example of a method for predicting the number of days until completion of treatment by using a plurality of prediction models. In the method exemplified in FIG. 4, the treatment period prediction unit 40 predicts the number of days until completion of treatment by using five prediction models. However, the number of prediction models to be used is not limited to five, but may be two to four, or may be six or more.

Each prediction model exemplified in FIG. 4 is a model for prediction as to whether or not treatment is to be completed within each of different periods to be predicted. For example, a prediction model 1 exemplified in FIG. 4 is a model for prediction as to whether or not the treatment period is within three days (that is, whether or not treatment is to be completed within three times), and a prediction model 2 is a model for prediction as to whether or not the treatment period is within one week.

In each prediction model exemplified in FIG. 4, a prediction difference according to a prediction result is determined in advance. For example, when the treatment period is predicted to be within three days (that is, when the result is predicted as “Yes” in the prediction model 1), the prediction difference is determined to be within one day, and when the treatment period is predicted to be within one week (that is, when the result is predicted to be “Yes” in the prediction model 2), the prediction difference is determined to be within four days. This is because the prediction difference is considered to be larger as the treatment period is predicted to be longer.

However, the method of setting the prediction difference can be optional, and is not limited to the number of days exemplified in FIG. 4. For example, magnitude of a predetermined prediction difference may be changed in accordance with learning results and accuracy of the prediction model.

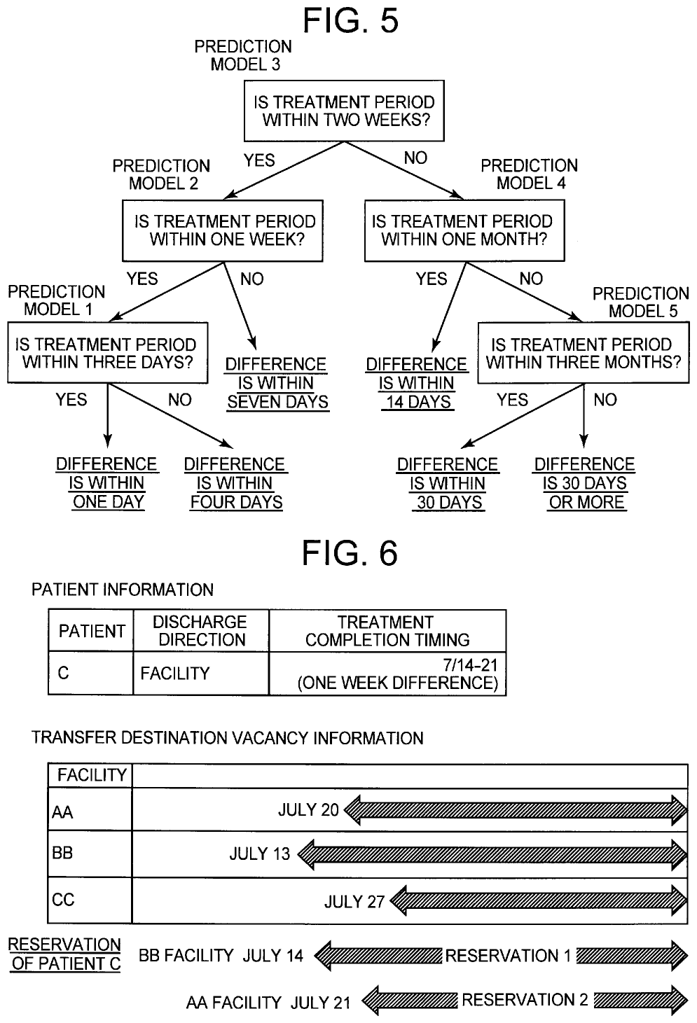

Further, FIG. 4 has exemplified a method for predicting the number of days until completion of treatment by sequentially increasing a predicted treatment period. However, the method for determining the treatment period is not limited to the method exemplified in FIG. 4. FIG. 5 is an explanatory view showing an example of another method for predicting the number of days until completion of treatment by using a plurality of prediction models. In the example exemplified in FIG. 5, a tree structure is assumed in which a prediction model 3 exemplified in FIG. 4 is arranged at a root node, and prediction models are sequentially selected in accordance with a prediction result. By using the structure exemplified in FIG. 5, it is possible to reduce the number of processes for determining the treatment period.

As described above, the treatment period prediction unit 40 predicts a treatment completion period on the basis of a plurality of prediction models for prediction as to whether or not treatment is to be completed within a predetermined period, and on the basis of a prediction difference determined in advance in accordance with a prediction result of the prediction model. Therefore, accuracy of estimating the number of days until completion of treatment can be improved.

The transfer destination extraction unit 50 extracts facility information satisfying a condition of a transfer destination of a patient from the transfer destination information storage unit 20. Specifically, the transfer destination extraction unit 50 extracts, from the transfer destination information storage unit 20, a facility that matches a type of a transfer destination (discharge direction) predicted by the discharge direction prediction unit 30. Further, in a case where the transfer destination facility determines a type of a patient whose reception is difficult, the transfer destination extraction unit 50 may exclude a facility for which the target patient matches the type of patient whose acceptance is difficult.

For example, it is assumed that a transfer destination of a patient C exemplified in FIG. 2 is extracted from the transfer destination information exemplified in FIG. 3. Further, as exemplified in FIG. 2, it is assumed that a discharge direction of the patient C is predicted to be “facility”. In this case, the transfer destination extraction unit 50 extracts a VV facility and a ZZ facility whose type is “facility”, from among the transfer destinations exemplified in FIG. 3. Further, since a state of the patient C is “disquiet”, the transfer destination extraction unit 50 excludes the ZZ facility in which “disquiet” is set to “NG patient”, from among the extracted VV facility and ZZ facility. As a result, the VV facility is extracted as a candidate for the transfer destination of the patient C.

Note that, in a case where a type of a transfer destination (discharge direction) predicted by the discharge direction prediction unit 30 cannot be completely specified, the transfer destination extraction unit 50 may extract a plurality of possible types of the transfer destination. For example, when a prediction of a rehabilitation hospital and a care hospital is even, the transfer destination extraction unit 50 may extract both types of the transfer destination.

In addition, in consideration of a place of residence of the patient and a place of residence of a caregiver of the patient, the transfer destination extraction unit 50 may use information on the target patient to extract information of a facility existing in an area where the patient lives or in the proximity thereof. Note that a degree of proximity may be determined in advance, such as neighboring municipalities or a distance.

The transfer destination determination unit 60 determines the transfer destination of the patient in accordance with the treatment completion period of the patient predicted by the treatment period prediction unit 40, from among the candidates for the transfer destination extracted by the transfer destination extraction unit 50. Specifically, the transfer destination determination unit 60 determines whether or not the operation status of the transfer destination facility indicates being capable of acceptance within the predicted treatment completion period. Then, on the day specified by the treatment completion period, the transfer destination determination unit 60 extracts a candidate for the transfer destination whose operation status indicates being capable of accepting the patient. The transfer destination determination unit 60 may, for example, extract a candidate for a transfer destination being capable of acceptance after the earliest treatment completion date in consideration of the prediction difference.

In addition, the transfer destination determination unit 60 may receive a patient's request, and limit the candidates for the transfer destination so as to match the received request. In this way, the transfer destination determination unit 60 determines a facility that satisfies a requirement of a transfer destination from among the facilities, based on the facility information that has been acquired and the transfer destination and the treatment completion period that have been predicted.

The transfer destination reservation unit 70 performs various processes for reserving facilities. In the following description, performing various processes for reserving a facility may be simply described as reserving the transfer destination facility. For example, when the transfer destination determination system cooperates with a facility reservation system (not shown), the transfer destination reservation unit 70 may notify a reservation target facility of a determination result. In addition, the transfer destination reservation unit 70 may output the determination result (for example, information on a transfer destination facility being capable of acceptance) to a display device, a printer device, or the like, or may transmit an email or the like to the transfer destination facility. At that time, the transfer destination reservation unit 70 may output the determination result in association with a treatment completion period. Hereinafter, a method for determining a facility to be reserved will be described.

For example, when the candidate for the transfer destination is determined to be one, the transfer destination reservation unit 70 may simply determine to reserve the transfer destination facility. Whereas, when there are a plurality of candidates for the transfer destination, the transfer destination reservation unit 70 may determine to reserve only one facility or may determine to reserve a plurality of facilities. For example, when the treatment completion period is long (the prediction difference is large), the transfer destination reservation unit 70 may preferentially select a facility that has allowance for acceptance (for example, a facility with many vacancies). By preferentially selecting such a facility, it is possible to reduce an influence of the prediction difference. Further, in consideration of a risk that a reservation cannot be made, the transfer destination reservation unit 70 may preferentially select a hospital with a small number of available vacancies or a busy hospital.

Any method for determining the number of reservations may be adopted. The transfer destination reservation unit 70 may, for example, determine the number of reservations in accordance with the number of transfer destinations requested by the patient, and increase the number of reservations in accordance with a length of the treatment completion period (magnitude of a prediction difference). In order to increase reliability of the reservation while reducing the number of reservations, the transfer destination reservation unit 70 may determine the facility to be reserved in accordance with the treatment completion period (a prediction difference).

FIG. 6 is an explanatory view showing an example of a facility reservation in consideration of a prediction difference. In the reservation method exemplified in FIG. 6, among the treatment completion timings in consideration of a prediction difference, a reservation taking into account that the patient moves on the earliest day is used as a first reservation (reservation 1), and a reservation taking into account that the patient moves on the latest day is used as a second reservation (reservation 2). Note that the dates to be considered for making the reservation 1 and the reservation 2 can be said to be timings at which the respective reservations are started, and thus can be referred to as reservation start timings.

For example, it is assumed that, as a result of using the prediction model 3 exemplified in FIG. 4, the treatment period prediction unit 40 predicts that the treatment period is within two weeks, and predicts that the prediction difference is seven days (within one week). As a result, it is assumed that the treatment completion timing of the patient C is predicted to be between July 14 and July 21, as exemplified in FIG. 6.

In this case, first, the transfer destination reservation unit 70 specifies a facility for which the reservation 1 can be made at the reservation start timing (that is, a facility being capable of acceptance before July 14). In the example shown in FIG. 6, only the facility BB has a vacancy before July 14 (that is, on July 13). Therefore, the transfer destination reservation unit 70 determines to make a first reservation for the facility BB.

Next, the transfer destination reservation unit 70 specifies a facility for which the reservation 2 can be made at the reservation start timing (that is, a facility being capable of acceptance before July 21). In the example shown in FIG. 6, the facilities AA and BB have vacancies before July 20. Since the first reservation is made for the facility BB, the transfer destination reservation unit 70 determines to make the second reservation for the facility AA.

Further, for example, when a reservation of another patient (for example, a reservation of a patient without a prediction difference) is made during a prediction difference period, the transfer destination reservation unit 70 may cancel the previous reservation (reservation 1), and prioritize the reservation of the another patient. In this case, it is possible to secure movement according to the second reservation while securing the reservation of the another patient.

Whereas, when no other reservation is made during the prediction difference period (that is, when a patient can be moved to the facility of the reservation 1), the transfer destination reservation unit 70 may simply determine that the later reservation (reservation 2) is to be canceled.

Note that FIG. 6 has exemplified an example of a method of making two reservations at the beginning and end of the prediction difference period. However, the number of reservations is not limited to two, and for example, the number of reservations may be increased when the prediction difference is large. Further, the number of reservations may be increased in accordance with the number of vacancies and a congestion degree at the transfer destination hospital.

Whereas, when there is no candidate for the transfer destination, the transfer destination reservation unit 70 may output an alternative. The transfer destination reservation unit 70 may output, for example, a transfer destination that is likely to have a vacancy at the earliest, or may output a transfer destination candidate that does not correspond to the user's request if there is a vacancy.

Further, in the present exemplary embodiment, a case has been described in which the transfer destination reservation unit 70 determines a plurality of facilities that satisfy a requirement of a transfer destination. This process may be performed by the transfer destination determination unit 60. For example, in the process exemplified in FIG. 6, the transfer destination determination unit 60 may determine the reservation start timing and the number of reservations on the basis of the treatment completion period. Then, the transfer destination determination unit 60 may determine a facility that satisfies a requirement of a transfer destination with a start point of the treatment completion period as the first reservation start timing, and may determine a facility that satisfies a requirement of a transfer destination with an end point of the treatment completion period as the second reservation start timing.

The discharge direction prediction unit 30, the treatment period prediction unit 40, the transfer destination extraction unit 50, the transfer destination determination unit 60, and the transfer destination reservation unit 70 are realized by a processor (for example, a central processing unit (CPU), a graphics processing unit (GPU), or a field-programmable gate array (FPGA)) of a computer that operates in accordance with a program (a transfer destination determination program). Further, the patient information storage unit 10 and the transfer destination information storage unit 20 are realized by, for example, a magnetic disk or the like.

The above-mentioned program may be stored in, for example, a storage unit (not shown). The processor may read the program, and operate, in accordance with the program, as the discharge direction prediction unit 30, the treatment period prediction unit 40, the transfer destination extraction unit 50, the transfer destination determination unit 60, and the transfer destination reservation unit 70. Further, a function of the transfer destination determination system may be provided in a software as a service (SaaS) format.

The discharge direction prediction unit 30, the treatment period prediction unit 40, the transfer destination extraction unit 50, the transfer destination determination unit 60, and the transfer destination reservation unit 70 each may be realized by dedicated hardware. In addition, part or all of each constituent element of each device may be realized by a general purpose or dedicated circuitry, a processor, or the like, or a combination thereof. These may be configured by a single chip or may be configured by a plurality of chips connected via a bus. Part or all of each constituent element of each device may be realized by a combination of the above-described circuitry and the like and a program.

Further, when part or all of each constituent element of the transfer destination determination system is realized by a plurality of information processing devices, circuitry, and the like, the plurality of information processing devices, circuitry, and the like may be arranged concentratedly or distributedly. For example, the information processing devices, the circuitry, and the like may be realized as a form in which each is connected via a communication network, such as a client server system, a cloud computing system, and the like.

Further, in the above description, processes of the transfer destination extraction unit 50, the transfer destination determination unit 60, and the transfer destination reservation unit 70 are described separately from each other, but the transfer destination extraction unit 50, the transfer destination determination unit 60, and the transfer destination reservation unit 70 may perform a process performed by another configuration. Since the transfer destination facility is determined by the transfer destination extraction unit 50, the transfer destination determination unit 60, and the transfer destination reservation unit 70, these configurations can be collectively referred to as a determination unit.

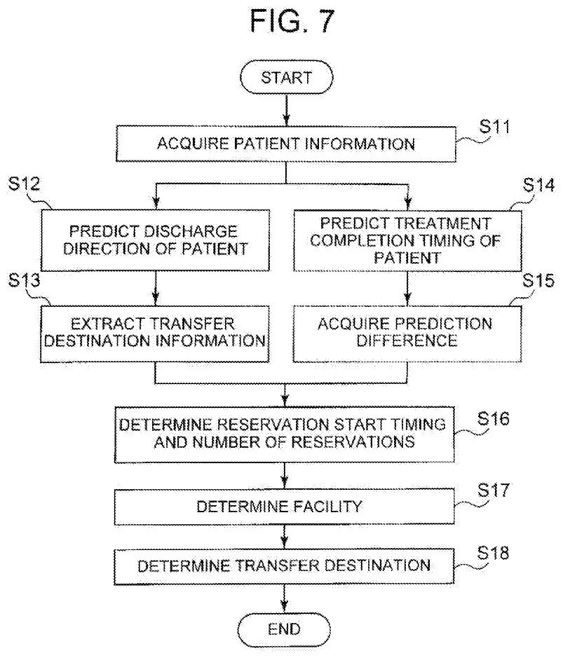

Next, an operation of the present exemplary embodiment will be described. FIG. 7 is a flowchart showing an operation example of the transfer destination determination system of the present exemplary embodiment. The discharge direction prediction unit 30 and the treatment period prediction unit 40 acquire patient information from the transfer destination information storage unit 20 (step S11).

The discharge direction prediction unit 30 predicts a discharge direction of a patient (step S12). Specifically, the discharge direction prediction unit 30 predicts a transfer destination on the basis of patient information and a first prediction model. Then, the transfer destination extraction unit 50 extracts transfer destination information from the transfer destination information storage unit 20 (step S13). Specifically, the transfer destination extraction unit 50 acquires facility information including an operation status of each facility from the transfer destination information storage unit 20.

Whereas, the treatment period prediction unit 40 predicts a treatment completion timing of the patient (step S14). Specifically, the treatment period prediction unit 40 predicts a treatment completion period on the basis of patient information and a second prediction model. Additionally, the treatment period prediction unit 40 acquires a prediction difference (step S15). Note that processing by the discharge direction prediction unit 30 (steps S12 and S13) and the processing by the treatment period prediction unit 40 (steps S14 and S15) may be performed sequentially or may be performed in parallel.

The transfer destination determination unit 60 determines a reservation start timing and the number of reservations on the basis of the treatment completion period (step S16). Then, on the basis of the acquired facility information and the predicted transfer destination and treatment completion period, the transfer destination determination unit 60 determines a facility that satisfies a requirement of the transfer destination (step S17). The transfer destination reservation unit 70 reserves the transfer destination facility on the basis of the determination result (step S18).

As described above, in the present exemplary embodiment, the discharge direction prediction unit 30 predicts a transfer destination of a target patient on the basis of inputted information on the target patient and the first prediction model, and the treatment period prediction unit 40 predicts a treatment completion period of the target patient on the basis of information on the target patient and the second prediction model. In addition, the transfer destination extraction unit 50 acquires facility information including an operation status of each facility. Then, on the basis of the acquired facility information and the predicted transfer destination and treatment completion period, the transfer destination determination unit 60 and the transfer destination reservation unit 70 determine a facility that satisfies a requirement of the transfer destination from among the facilities, and output a result of the determination. Therefore, the transfer destination can be determined so as to shorten a hospital stay duration of the patient.

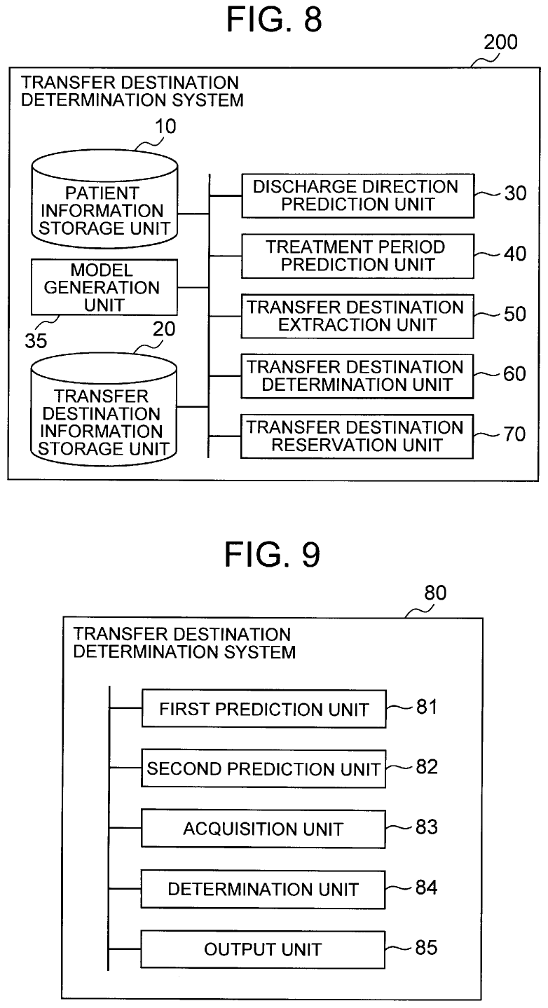

Next, a modified example of the present exemplary embodiment will be described. FIG. 8 is a block diagram showing a modified example of the transfer destination determination system according to the present invention. A transfer destination determination system 200 exemplified in FIG. 8 includes a patient information storage unit 10, a transfer destination information storage unit 20, a discharge direction prediction unit 30, a model generation unit 35, a treatment period prediction unit 40, a transfer destination extraction unit 50, a transfer destination determination unit 60, and a transfer destination reservation unit 70. That is, the transfer destination determination system according to the present modified example is different from the above-described exemplary embodiment in that the model generation unit 35 is provided.

The model generation unit 35 generates a model (a first model and a second model) to be used by the discharge direction prediction unit 30 and the treatment period prediction unit 40 for prediction. Specifically, the model generation unit 35 generates a prediction model by performing machine learning on electronic medical chart data of a plurality of patients. Note that the model generation unit 35 may generate only one of the first model and the second model, or may generate both models.

The model generation unit 35 may learn a prediction model on the basis of a value (item value) of each data item of the electronic medical chart. More specifically, when learning a model for prediction of a treatment completion period (that is, a second prediction model), the model generation unit 35 may use, for example, data of the patient information exemplified in FIG. 2, as learning data. Further, when learning a model for prediction of a transfer destination (that is, a first prediction model), the model generation unit 35 may use, as learning data, data indicating information on the transfer destination, in addition to the patient information exemplified in FIG. 2.

Examples of data indicating the transfer destination information include: a hospital name, a hospital type (acute, recovery (rehabilitation), medical care, and the like), an address (a distance from a hospital of current hospitalization), the number of beds, the number of vacant beds (for example, the number of vacant beds up to one week ahead), a non-acceptable patient (disquiet or tube-fed patients and the like), and a transfer history (how many patients have been transferred from the hospital of current hospitalization).

When there is a model generated by the model generation unit 35, the discharge direction prediction unit 30 and the treatment period prediction unit 40 use the model to perform each prediction. Other operations are the same as those in the above exemplary embodiment.

Next, an outline of the present invention will be described. FIG. 9 is a block diagram showing an outline of a transfer destination determination system according to the present invention. A transfer destination determination system 80 according to the present invention includes: a first prediction unit 81 (for example, the discharge direction prediction unit 30) that predicts a transfer destination of a target patient, based on inputted information on the target patient (for example, patient information stored in the patient information storage unit) and a first prediction model for prediction of a transfer destination of a patient; a second prediction unit 82 (for example, the treatment period prediction unit 40) that predicts a treatment completion period of the target patient, based on information on the target patient and a second prediction model for prediction of a treatment completion period of a patient; an acquisition unit 83 (for example, the transfer destination extraction unit 50) that acquires facility information including an operation status of each facility; a determination unit 84 (for example, the transfer destination determination unit 60) that determines a facility that satisfies a requirement of a transfer destination from among the facilities, based on the facility information that has been acquired and the transfer destination and the treatment completion period that have been predicted; and an output unit 85 (for example, the transfer destination reservation unit 70) that outputs a result determined by the determination unit 84.

Such a configuration enables a transfer destination to be determined so as to shorten a hospital stay duration of the patient.

Specifically, the determination unit 84 may determine whether or not an operation status of the transfer destination facility indicates being capable of acceptance within the treatment completion period.

Further, the second prediction unit 82 may predict the treatment completion period on the basis of a plurality of prediction models for prediction as to whether or not treatment is to be completed within a predetermined period, and on the basis of a prediction difference determined in advance in accordance with a prediction result of the prediction model. According to such a configuration, a system for prediction of a treatment completion period can be improved.

In addition, the determination unit 84 may determine to more preferentially select a facility having allowance for acceptance, as the predicted treatment completion period is longer. It is more difficult to determine the timing to move to the transfer destination facility as the predicted treatment completion period is longer. Therefore, by preferentially selecting a facility that has allowance, the risk of rejection of a reservation can be avoided.

In addition, the determination unit 84 may determine to reserve more facilities as the predicted treatment completion period is longer. As described above, it is more difficult to determine the timing to move to the transfer destination facility as the predicted treatment completion period is longer. Therefore, by reserving more facilities, the risk of being unable to make a reservation can be avoided.

Specifically, the determination unit 84 may determine a facility that satisfies a requirement of the transfer destination with a start point of the treatment completion period as a first reservation start timing, and may determine a facility that satisfies a requirement of the transfer destination with an end point of the treatment completion period as a second reservation start timing. This makes it possible to increase reliability of the reservation while reducing the number of reservations.

In addition, the acquisition unit 83 may use information on the target patient to acquire information on a facility existing in an area where the target patient lives. This makes it possible to reduce a burden on the patient for visiting the facility.

Further, the transfer destination determination system 80 may include a model generation unit (for example, the model generation unit 35) that generates a second prediction model by performing machine learning on electronic medical chart data of a plurality of patients.

Some or all of the above exemplary embodiments may be described as in the following supplementary notes, but are not limited to the following.

(Supplementary note 1) A transfer destination determination system including: a first prediction unit that predicts a transfer destination of a target patient, based on inputted information on the target patient and a first prediction model for prediction of a transfer destination of a patient; a second prediction unit that predicts a treatment completion period of the target patient, based on information on the target patient and a second prediction model for prediction of a treatment completion period of a patient; an acquisition unit that acquires facility information including an operation status of each facility; a determination unit that determines a facility that satisfies a requirement of a transfer destination from among the facilities, based on the facility information that has been acquired and the transfer destination and the treatment completion period that have been predicted; and an output unit that outputs a result determined by the determination unit.

(Supplementary note 2) The transfer destination determination system according to Supplementary note 1, in which the determination unit determines whether or not an operation status of a transfer destination facility indicates being capable of acceptance within a treatment completion period.

(Supplementary note 3) The transfer destination determination system according to Supplementary note 1 or 2, in which the second prediction unit predicts a treatment completion period, based on a plurality of prediction models for prediction as to whether or not treatment is to be completed within a predetermined period, and based on a prediction difference determined in advance in accordance with a prediction result of each of the prediction models.

(Supplementary note 4) The transfer destination determination system according to any one of Supplementary notes 1 to 3, in which the determination unit determines to more preferentially select a facility having allowance for acceptance as a predicted treatment completion period is longer.

(Supplementary note 5) The transfer destination determination system according to any one of Supplementary notes 1 to 4, in which the determination unit determines to reserve more facilities as a predicted treatment completion period is longer.

(Supplementary note 6) The transfer destination determination system according to any one of Supplementary notes 1 to 5, in which the determination unit determines a facility that satisfies a requirement of a transfer destination with a start point of a treatment completion period as a first reservation start timing, and determines a facility that satisfies a requirement of a transfer destination with an end point of a treatment completion period as a second reservation start timing.

(Supplementary note 7) The transfer destination determination system according to any one of Supplementary notes 1 to 6, in which the acquisition unit uses information on a target patient to acquire information on a facility existing in an area where the target patient lives.

(Supplementary note 8) The transfer destination determination system according to any one of Supplementary notes 1 to 7, further including a model generation unit that generates a second prediction model by performing machine learning on electronic medical chart data of a plurality of patients.

(Supplementary note 9) The transfer destination determination system according to any one of Supplementary notes 1 to 8, in which the acquisition unit uses information on a target patient to acquire information on a facility existing in an area where a caregiver of the target patient lives.

(Supplementary note 10) The transfer destination determination system according to any one of Supplementary notes 1 to 9, in which

the output unit outputs a determination result and a treatment completion period in association with each other.

(Supplementary note 11) A transfer destination determination method including: predicting a transfer destination of a target patient, based on inputted information on the target patient and a first prediction model for prediction of a transfer destination of a patient; predicting a treatment completion period of the target patient, based on information on the target patient and a second prediction model for prediction of a treatment completion period of a patient; acquiring facility information including an operation status of each facility; determining a facility that satisfies a requirement of a transfer destination from among the facilities, based on the facility information that has been acquired and the transfer destination and the treatment completion period that have been predicted; and outputting a determined result.

(Supplementary note 12) The transfer destination determination method according to Supplementary note 11, in which it is determined whether or not an operation status of a transfer destination facility indicates being capable of acceptance within a treatment completion period.

(Supplementary note 13) A transfer destination determination program for causing a computer to execute: a first prediction process of predicting a transfer destination of a target patient, based on inputted information on the target patient and a first prediction model for prediction of a transfer destination of a patient; a second prediction process of predicting a treatment completion period of the target patient, based on information on the target patient and a second prediction model for prediction of a treatment completion period of a patient; an acquisition process of acquiring facility information including an operation status of each facility; a determination process of determining a facility that satisfies a requirement of a transfer destination from among the facilities, based on the facility information that has been acquired and the transfer destination and the treatment completion period that have been predicted; and an output process of outputting a result determined in the determination process.

(Supplementary note 14) The transfer destination determination program according to Supplementary note 13, in which a computer is caused to determine whether or not an operation status of a transfer destination facility indicates being capable of acceptance within a treatment completion period in the determination process.

Although the present invention has been described with reference to the exemplary embodiments and examples, the present invention is not limited to the above exemplary embodiments and examples. Various changes that can be understood by those skilled in the art can be made to the configuration and details of the present invention within the scope of the present invention.

This application claims priority based on Japanese Patent Application No. 2017-200719, filed on Oct. 17, 2017, the entire disclosure of which is incorporated herein.

### REFERENCE SIGNS LIST

10 Patient information storage unit
20 Transfer destination information storage unit
30 Discharge direction prediction unit
35 Model generation unit
40 Treatment period prediction unit
50 Transfer destination extraction unit
60 Transfer destination determination unit
70 Transfer destination reservation unit
100 Transfer destination determination system

1. A transfer destination determination system comprising a hardware processor configured to execute a software code to:
predict a transfer destination of a target patient, based on inputted information on the target patient and a first prediction model for prediction of a transfer destination of a patient;
predict a treatment completion period of the target patient, based on information on the target patient and a second prediction model for prediction of a treatment completion period of a patient;
acquire facility information including an operation status of each facility;
determine a facility that satisfies a requirement of a transfer destination from among the facilities, based on the facility information, the transfer destination, and the treatment completion period; and
output a determined result.

2. The transfer destination determination system according to claim 1, wherein the hardware processor is configured to execute a software code to determine whether or not an operation status of a transfer destination facility indicates being capable of acceptance within a treatment completion period.

3. The transfer destination determination system according to claim 1, wherein the hardware processor is configured to execute a software code to predict a treatment completion period, based on a plurality of prediction models for prediction as to whether or not treatment is to be completed within a predetermined period, and based on a prediction difference determined in advance in accordance with a prediction result of each of the prediction models.

4. The transfer destination determination system according to claim 1, wherein
the determination unit determines to more preferentially select a facility having allowance for acceptance as a predicted treatment completion period is longer.

5. The transfer destination determination system according to claim 1, wherein the hardware processor is configured to execute a software code to determine to reserve more facilities as a predicted treatment completion period is longer.

6. The transfer destination determination system according to claim 1, wherein the hardware processor is configured to execute a software code to determine a facility that satisfies a requirement of a transfer destination with a start point of a treatment completion period as a first reservation start timing, and determine a facility that satisfies a requirement of a transfer destination with an end point of a treatment completion period as a second reservation start timing.

7. The transfer destination determination system according to claim 1, wherein the hardware processor is configured to execute a software code to use information on a target patient to acquire information on a facility existing in an area where the target patient lives.

8. The transfer destination determination system according to claim 1, wherein the hardware processor is configured to generate a second prediction model by performing machine learning on electronic medical chart data of a plurality of patients.

9. The transfer destination determination system according to claim 1, wherein the hardware processor is configured to use information on a target patient to acquire information on a facility existing in an area where a caregiver of the target patient lives.

10. The transfer destination determination system according to claim 1, wherein the hardware processor is configured to output a determination result and a treatment completion period in association with each other.

11. A transfer destination determination method comprising:
predicting a transfer destination of a target patient, based on inputted information on the target patient and a first prediction model for prediction of a transfer destination of a patient;
predicting a treatment completion period of the target patient, based on information on the target patient and a second prediction model for prediction of a treatment completion period of a patient;
acquiring facility information including an operation status of each facility;
determining a facility that satisfies a requirement of a transfer destination from among the facilities, based on the facility information that has been acquired and the transfer destination and the treatment completion period that have been predicted; and
outputting a determined result.

12. The transfer destination determination method according to claim 11, wherein
it is determined whether or not an operation status of a transfer destination facility indicates being capable of acceptance within a treatment completion period.

13. A non-transitory computer readable information recording medium storing a transfer destination determination program, when executed by a processor, that performs a method for:
predicting a transfer destination of a target patient, based on inputted information on the target patient and a first prediction model for prediction of a transfer destination of a patient;
predicting a treatment completion period of the target patient, based on information on the target patient and a second prediction model for prediction of a treatment completion period of a patient;
acquiring facility information including an operation status of each facility;
determining a facility that satisfies a requirement of a transfer destination from among the facilities, based on the facility information that has been acquired and the transfer destination and the treatment completion period that have been predicted; and
outputting a determined result.

14. The non-transitory computer readable information recording medium according to claim 13, wherein
it is determined whether or not an operation status of a transfer destination facility indicates being capable of acceptance within a treatment completion period.
